(19) United States
(12) Patent Application Publication
(10) Pub. No.: US 2013/0073020 A1
(43) Pub. Date: Mar. 21, 2013

(54) HEADER EMBEDDED FILTER FOR IMPLANTABLE MEDICAL DEVICE

(71) Applicants: Gabriel A. Mouchawar, Valencia, CA (US); Ramez Shehada, La Mirada, CA (US); Xiaoyi Min, Thousand Oaks, CA (US)

(72) Inventors: Gabriel A. Mouchawar, Valencia, CA (US); Ramez Shehada, La Mirada, CA (US); Xiaoyi Min, Thousand Oaks, CA (US)

(73) Assignee: PACESETTER, INC., Sylmar, CA (US)

(21) Appl. No.: 13/233,966

(22) Filed: Sep. 15, 2011

(51) Int. Cl. A61N 1/00

(52) U.S. Cl. 607/116

(57) ABSTRACT

A filter circuit embedded into a header of an implantable medical device attenuates energy that may otherwise enter the implantable medical device. At MRI frequencies, the impedance of the filter circuit is much higher than the impedance of the feedthrough capacitor of the implantable medical device. Thus, MRI-induced current that would otherwise enter the implantable medical device is limited by the filter circuit. Consequently, localized device heating that may otherwise occur during MRI scanning is significantly reduced by operation of the filter circuit. In some implementations, the header embedded filter circuit is electrically isolated from the header housing. In this way, localized heating of the header housing also may be avoided.

### TECHNICAL FIELD

This application relates generally to implantable medical devices and more specifically, but not exclusively, to a header embedded filter for an implantable medical device.

### BACKGROUND

Some types of implantable medical devices connect to one or more implantable leads. For example, an implantable cardiac rhythm management device (e.g., a pacemaker, a defibrillator, or a cardioverter defibrillator) typically connects to one or more leads implanted in or near the heart of a patient who suffers from cardiac arrhythmia to monitor cardiac function and provide therapy for the patient. As another example, a neurostimulator typically connects to one or more leads implanted in or near nervous system tissue of a patient to monitor neurological function and provide appropriate therapy for the patient.

An implantable medical device and associated implantable lead are prone to heating and induced current when placed in strong electromagnetic (static, gradient and radiofrequency (RF)) fields of a magnetic resonance imaging (MRI) machine. In the electromagnetic field of the MRI machine, the lead acts as an antenna and picks up RF currents that can cause heating at either end of the lead. Currents exiting the distal end (e.g., at the tip or tissue electrode) of the lead may directly heat patient tissue.

Currents exiting the proximal end of the lead (e.g., via a connector of the implantable medical device) may enter the device and cause heating of the device and, consequently, cause heating of tissue surrounding the device. For example, RF energy at MRI frequencies may encounter very low impedance (e.g., on the order of a few ohms) at the feedthrough capacitor employed in some implantable medical devices. Consequently, substantial heating may occur at the feedthrough capacitor and in the vicinity of the feedthrough capacitor.

In addition, the presence of induced RF signals of a relative large magnitude at an input or output terminal of an implantable medical device may result in rectification of the signal by internal circuitry of the device. Under certain circumstances, the resulting rectified signal causes asynchronous stimulation which may, in turn induce arrhythmia in the patient.

In view of the above, MRI-induced heating and rectification are undesirable and should be avoided. Thus, a need exists for implantable medical devices that are sufficiently immune to the influence of MRI magnetic fields and other similar electromagnetic interference (EMI).

### SUMMARY

A summary of several sample aspects of the disclosure follows. It should be appreciated that this summary is provided for the convenience of the reader and does not wholly define the breadth of the disclosure. One or more aspects or embodiments of the disclosure may be referred to herein simply as “some aspects” or “some embodiments.”

The disclosure relates in some aspects to a filter circuit for mitigating potentially adverse effects that may result from an implantable medical device being subjected to EMI. For example, a filter circuit as taught herein is used in some implementations to mitigate potentially adverse heating and rectification effects that would otherwise be caused by MRI scanning of a patient that has an implantable medical device.

In a typical scenario, the filter circuit is used to limit RF currents that are induced on one or more leads that are connected to an implantable medical device. In some implementations, the filter circuit comprises a low pass filter that has a high attenuation at MRI frequencies (e.g., 64 MHz for a 1.5 Tesla MRI scanner and 128 MHz for a 3 Tesla MRI scanner) and therefore limits current flow associated with those frequencies. In other implementations, the filter circuit comprises a band pass filter that has a high attenuation at MRI frequencies.

In some implementations, a filter circuit as taught herein is embedded into a header of an implantable medical device. At MRI frequencies, the impedance of the filter circuit is much higher than the impedance of the feedthrough capacitor of the implantable medical device. Consequently, MRI-induced energy that would otherwise enter the implantable medical device and be dissipated by the feedthrough capacitor of the implantable medical device is attenuated by the filter circuit in the header. As a result, localized device heating (e.g., at the feedthrough capacitor) that would otherwise occur during MRI scanning is significantly reduced by operation of the filter circuit embedded in the device header.

In some implementations, a header-based filter circuit is electrically isolated from the header housing. Localized heating of the header housing that could otherwise result from dissipating energy into the housing may thus be avoided, thereby eliminating potential hot spots that could otherwise damage patient tissue that is in contact with the header housing.

In some aspects, a header-based filter circuit is advantageously employed in conjunction with an implantable lead-based filter circuit. For example, a filter circuit located at a distal portion of a lead is employed to prevent RF energy induced on the lead from causing heating at the point where a distal electrode of the lead contacts the tissue. The header-based filter circuit is then employed to prevent most of this RF energy from instead passing through the feedthrough capacitor of the implantable medical device.

The filter circuit attenuates MRI frequency signal energy without significantly affecting signals (e.g., intrinsic cardiac signals) that the implantable medical device is intended to sense and without significantly affecting stimulation signals (e.g., cardiac pacing signals) generated by the implantable medical device. This is achieved by designing the filter circuit to provide high impedance at the higher frequencies associated with, for example, the MRI-induced signals and provide low impedance at the lower frequencies associated with sensing and stimulation (e.g., cardiac sensing and/or cardiac pacing).

### BRIEF DESCRIPTION OF THE DRAWINGS

These and other features, aspects, and advantages will be more fully understood when considered with respect to the following detailed description, the appended claims, and the accompanying drawings, wherein:

FIG. 1 is a simplified schematic diagram of an embodiment of an implantable medical device comprising a filter circuit embedded in a device header;

FIG. 2 is a simplified diagram of an embodiment of an implantable medical device comprising a filter circuit embedded in a device header;

FIG. 3 is a simplified schematic diagram of an embodiment of an implantable medical device where header-based filter circuits are employed for multiple lead conductors;

FIG. 4 is a simplified schematic diagram of an embodiment of an implantable medical system where header-based filter circuits and lead-based filter circuits are employed for multiple lead conductors;

FIG. 5A is a simplified schematic diagram of an embodiment of an implantable medical system where different types of filter circuits are employed for multiple lead conductors;

FIG. 5B is a simplified schematic diagram of another embodiment of an implantable medical system where different types of filter circuits are employed for multiple lead conductors;

FIG. 6 is a simplified schematic diagram of an embodiment of an implantable medical system where filter circuits are employed on distal and proximal ends of a lead;

FIG. 7 is a simplified diagram of another embodiment of an implantable medical device comprising a filter circuit embedded in a device header;

FIG. 8 is a simplified diagram of an embodiment of an implantable cardiac device in electrical communication with one or more leads implanted in a patient's heart for sensing conditions in the patient, delivering therapy to the patient, or providing some combination thereof; and

FIG. 9 is a simplified functional block diagram of an embodiment of an implantable cardiac device, illustrating basic elements that may be configured to sense conditions in the patient, deliver therapy to the patient, or provide some combination thereof.

In accordance with common practice the various features illustrated in the drawings may not be drawn to scale. Accordingly, the dimensions of the various features may be arbitrarily expanded or reduced for clarity. In addition, some of the drawings may be simplified for clarity. Thus, the drawings may not depict all of the components of a given apparatus or method. Finally, like reference numerals may be used to denote like features throughout the specification and figures.

### DETAILED DESCRIPTION

The description that follows sets forth one or more illustrative embodiments. It will be apparent that the teachings herein may be embodied in a wide variety of forms, some of which may appear to be quite different from those of the disclosed embodiments. Consequently, the specific structural and functional details disclosed herein are merely representative and do not limit the scope of the disclosure. For example, based on the teachings herein one skilled in the art should appreciate that the various structural and functional details disclosed herein may be incorporated in an embodiment independently of any other structural or functional details. Thus, an apparatus may be implemented or a method practiced using any number of the structural or functional details set forth in any disclosed embodiment(s). Also, an apparatus may be implemented or a method practiced using other structural or functional details in addition to or other than the structural or functional details set forth in any disclosed embodiment(s).

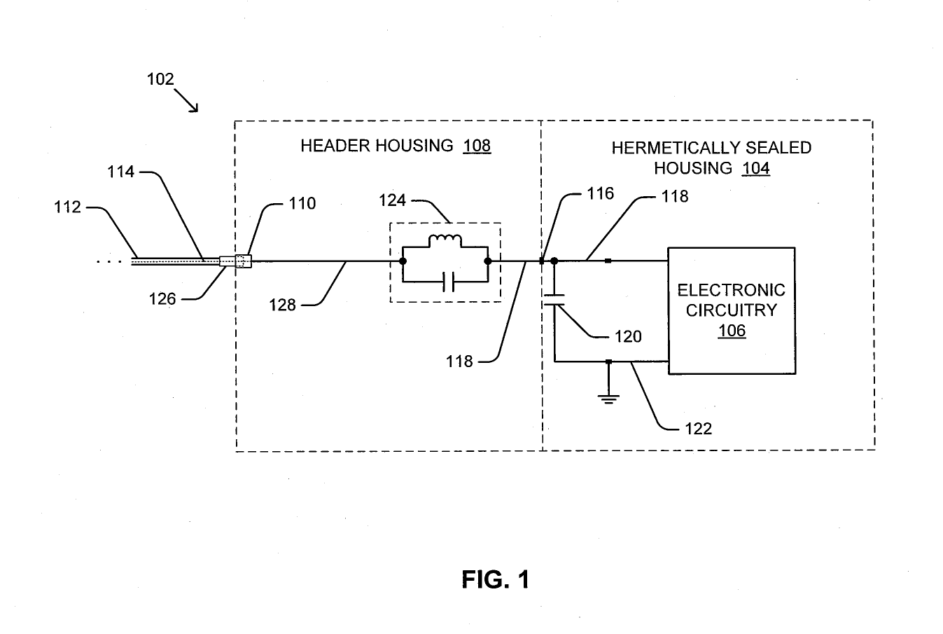

FIG. 1 is a simplified schematic diagram of an embodiment of an implantable medical device 102. The implantable medical device 102 provides different functionality in different implementations. In some implementations, the implantable medical device 102 is a cardiac device configured for stimulating cardiac tissue and/or sensing cardiac activity. For example, the implantable medical device 102 may be a pacemaker, a defibrillator, or an implantable cardioverter defibrillator (ICD), or some other type of implantable cardiac device. In some implementations, the implantable medical device 102 is a neurological device configured for stimulating nervous system tissue and/or sensing neurological activity.

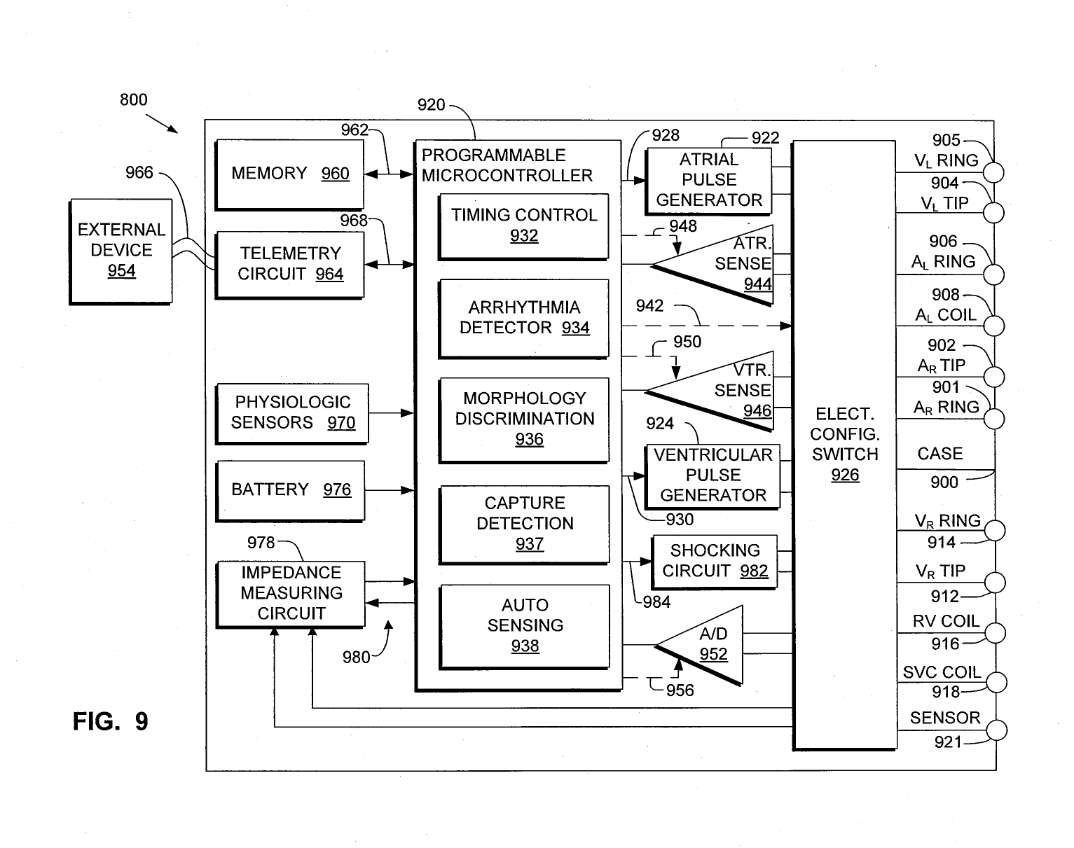

The device 102 includes a hermetically sealed housing 104 (represented by the right-hand-side dashed box) that houses electronic circuitry 106 that provides desired medical functionality. An example of this functionality is described in detail below in conjunction with FIG. 9 for an implantable cardiac device implementation. To enable the device 102 to be implanted within a patient, the housing 104 is made of a material suitable for implant (e.g., titanium) and is hermetically sealed to isolate internal components from patient tissue and fluid.

The device 102 also includes a header housing 108 that houses one or more connectors (hereafter referred to, for convenience, as the connector 110) that allow one or more implantable leads (hereafter referred to, for convenience, as the lead 112) to connect to the device 102. In particular, the connector 110 couples one or more conductors 114 of the lead 112 to internal circuitry of the device 102. Again, to enable the device 102 to be implanted within a patient, the housing 106 is made of a material suitable for implant and is hermetically sealed to isolate internal components from patient tissue and fluid.

The housing 104 includes at least one hermetically sealed feedthrough 116 that maintains the hermitical seal of the housing 104 while enabling the coupling of signals between the lead 112 and the circuitry 106. Here, one or more conductors (hereafter referred to, for convenience, as the conductor 118) are routed through the feedthrough 116 between the interior of the housing 104 and the interior of the housing 108.

A feedthrough capacitor 120 is provided in proximity to (e.g., within or near) the feedthrough 116 for preventing high frequency signals from entering the housing 104. In a typical implementation, the feedthrough capacitor 120 is coupled to a ground path 122 provided by the housing 104. The feedthrough capacitor 120 thus provides a layer of protection against EMI by shunting high frequency current directly to the housing 104 and away from the circuitry 106 where it could otherwise cause undesirable effects as discussed above. Being physically located at the point where the conductor 118 enters the housing 104, the feedthrough capacitor 120 shunts EMI-induced current away from the conductor 118 before or as the conductor 118 enters the housing 104, thereby reducing the potential for high frequency energy to re-radiate from the conductor 118 within the housing, possibly causing additional undesirable effects.

In a typical implementation, the feedthrough capacitor 120 has a value on the order of a few nanofarads. Thus, at MRI frequencies, the feedthrough capacitor 120 has an impedance on the order of a few ohms or less. Accordingly, the feedthrough capacitor 120 could incur significant heating if it is subjected to a relatively high level of inbound MRI-induced RF energy.

In accordance with the teachings herein, a filter circuit 124 is mounted within the housing 108 to limit RF currents collected by the lead 112. For example, an MRI-induced signal on the conductor 114 is coupled via a connector 126 of the lead to the connector 110 mounted within the housing 108 and then to one or more conductors (hereafter referred to, for convenience, as the conductor 128) within the housing 108. The conductor 128 is coupled to the filter circuit 124 which has relatively high impedance at MRI frequencies and, therefore, limits the amount of current that will flow on the conductor 118 and into the housing 104. In a sample embodiment, at MRI scanning frequencies, the impedance of the filter circuit 124 is at least an order of magnitude greater than the impedance of the feedthrough capacitor 120. For example, the filter circuit 124 may have an impedance of 200 ohms while the feedthrough capacitor has an impedance of 2 ohms. In this case, virtually all of the energy of the MRI-induced signal is dissipated by the filter circuit 124.

The filter circuit 124 is implemented in different ways in different implementations. In some implementations, the filter circuit 124 is a band stop filter that is configured to attenuate MRI-induced RF energy. In some aspects, the band stop circuit may have a resonant frequency that corresponds to an MRI scanning frequency. For example, the band stop filter may be tuned to have a relatively high attenuation at frequencies in the range of approximately 64 MHz and/or 128 MHz. Typically, the filter circuit 124 comprises an inductor-capacitor (L-C) circuit. As illustrated in FIG. 1, in some cases, the band stop filter comprises an L-C tank circuit. In some cases, the band stop filter comprises a self resonating inductor.

In some implementations, the filter circuit 124 is a low pass filter (e.g., an inductor). The low pass filter is configured to have a relatively high attenuation (e.g., 200 ohms or more) in the range of approximately 64 MHz and/or 128 MHz.

The filter circuit 124 (e.g., a band stop filter or a low pass filter) is configured to have relatively low impedance at the frequencies associated with signals that are sensed or generated by the circuitry 106 during normal operation. For example, for a cardiac device, sensed cardiac signals and stimulation signal typically are associated with frequencies on the order of kilohertz or less.

In some aspects, the filter circuit 124 is implemented to reduce localized heating of the device 102. For example, in some implementations, the filter circuit 124 is sized to facilitate even heat distribution over a relatively large area.

In some implementations, the filter circuit 124 is electrically isolated from the housing 108 (e.g., in a case where the housing is conductive). Thus, in this case, energy from inbound MRI-induced signals is not directed (e.g., via a ground path) to the housing 108 and/or the housing 104. Thus, localized heating may be mitigated in this case in contrast to implementations where such current is directed to a specific area (e.g., a solder terminal on a housing) of the device 102.

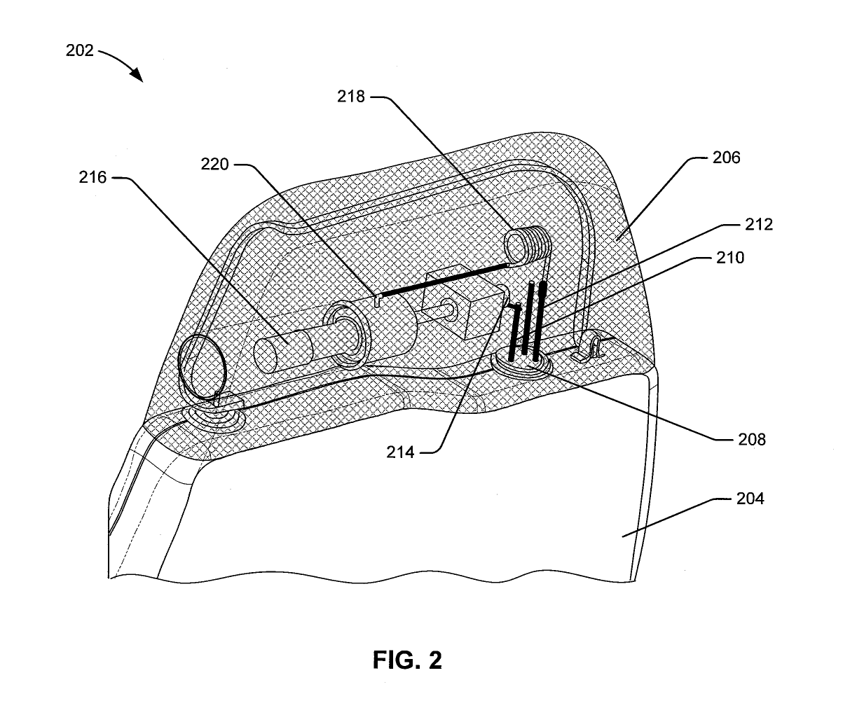

FIG. 2 illustrates an example of how a filter circuit may be deployed within a header between a feedthrough and a lead connector. In this example, an implantable medical device 202 comprises a main body 204 (shown in a partial view) and a header 206 (shown by the crosshatching and illustrating internal circuitry). With reference to FIG. 1, the main body 204 corresponds to the housing 104 and associated internal circuitry, while the header 206 corresponds to the housing 108 and associated internal circuitry. A hermetically sealed feedthrough 208 is provided in the housing of the main body 204. Several conductors (e.g., conductors 210 and 212) pass through the feedthrough 208.

The conductor 210 is coupled to a terminal 214 of a connector 216. For example, upon insertion of an implantable lead (not shown in FIG. 2) into the connector 216, the terminal 214 connects to a center conductor of the lead.

The conductor 212 is coupled to one terminal of a filter circuit 218 (e.g., an inductor or a self resonating inductor). Another terminal of the filter circuit 218 is coupled to a terminal 220 of the connector 216. For example, upon insertion of an implantable lead into the connector 216, the terminal 220 connects to an outer conductor of the lead. Thus, MRI-induced energy carried by the outer conductor of the lead will be attenuated by the filter circuit 218 before this energy reaches the feedthrough 208.

An inductor of the filter circuit 218 may be implemented in various ways. In some implementations, such an inductor takes the form of a bobbin inductor (e.g., comprising insulated wire wound on the bobbin). In cases where multiple inductors are employed in the header, insulated wires for each of the inductors may be wound in parallel on a bobbin to save space within the header.

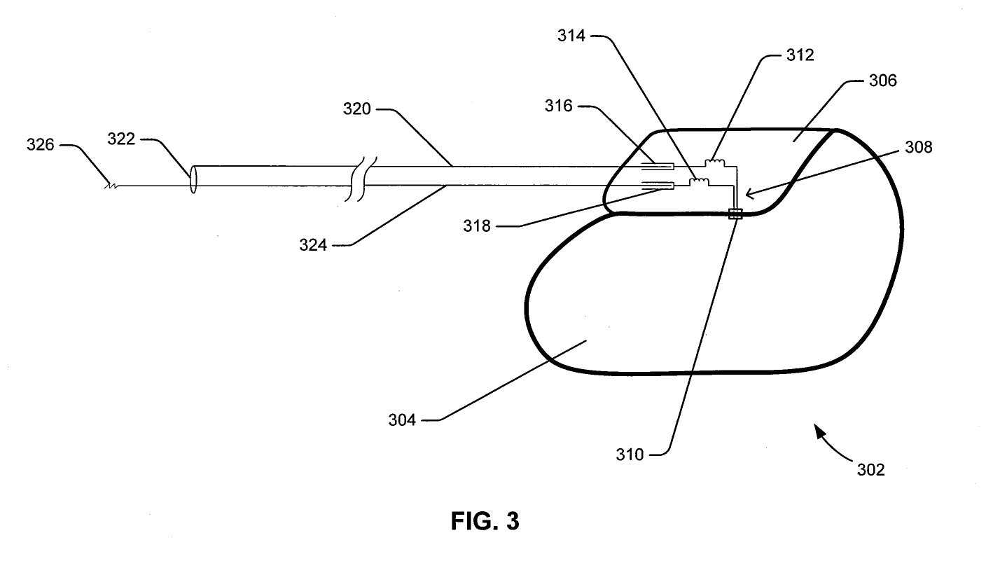

A filter circuit as taught herein may be employed in different locations in different implementations. FIGS. 3-6 illustrate three sample implementations.

In some implementations, multiple filter circuits are employed in a header. FIG. 3 illustrates a simplified schematic diagram of an implantable medical system where a different filter circuit is employed for each conductor of an implantable lead (e.g., an implantable cardiac lead). An implantable medical device 302 (e.g., an implantable cardiac device) comprises a main body 304 and a header 306. A pair of conductors 308 passes through a feedthrough 310 and into the header 306. A first one of the conductors 308 is coupled to a first filter circuit 312 and a second one of the conductors 308 is coupled to a second filter circuit 314. The first filter circuit 312 is coupled by a first connector terminal 316 to a ring conductor 320 of an implantable lead. The distal end of the ring conductor 318 is coupled to a ring electrode 322 of the lead. The second filter circuit 314 is coupled by a second connector terminal 318 to a tip conductor 324 of the implantable lead. The distal end of the tip conductor 324 is coupled to a tip electrode 326 (e.g., a helix electrode) of the lead. Thus, in the implementation of FIG. 3, MRI-induced energy carried by either the ring conductor 320 or the tip conductor 324 will be attenuated by the filter circuit 312 or the filter circuit 314, respectively, prior to the feedthrough 310. Consequently, this energy will not cause significant heating or rectification at the device 302.

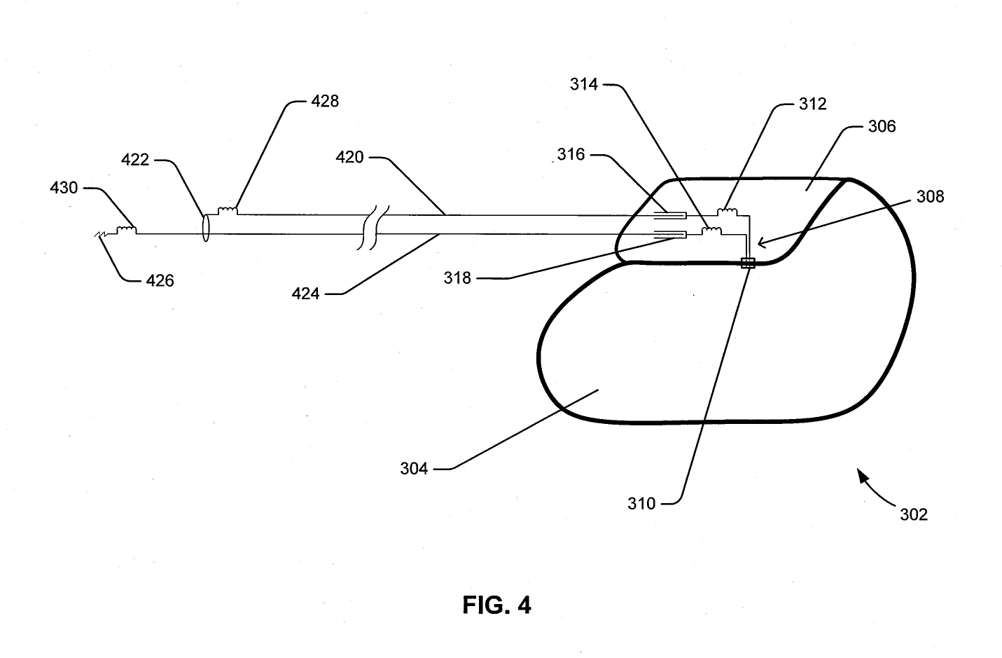

In some implementations, a filter circuit is employed at the distal end of one or more conductors of an implantable lead. FIG. 4 illustrates a simplified schematic diagram where the implantable medical device 302 of FIG. 3 is connected to a lead that employs two filter circuits for two lead conductors. In this example, a filter circuit 428 is provided near the ring electrode 422 of the ring conductor 420. In addition, a filter circuit 430 is provided near the tip electrode 426 of the tip conductor 424.

In the implementation of FIG. 4, MRI-induced energy carried by the ring conductor 420 is attenuated by the filter circuit 428 prior to entering the ring electrode 422. Similarly, MRI-induced energy carried by the tip conductor 424 is attenuated by the filter circuit 430 prior to entering the tip electrode 426. Thus, heating of tissue at the electrode implant locations will be significantly reduced or entirely eliminated. Moreover, the MRI-induced energy will not cause significant heating or rectification at the device 302 due to the operation of the filter circuit 312 and the filter circuit 314 as discussed above.

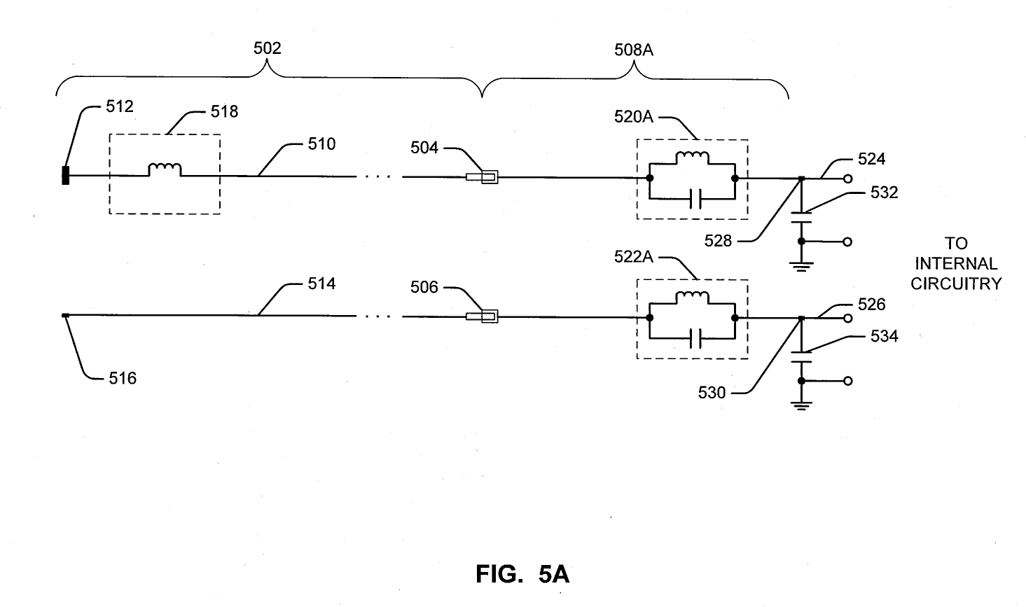

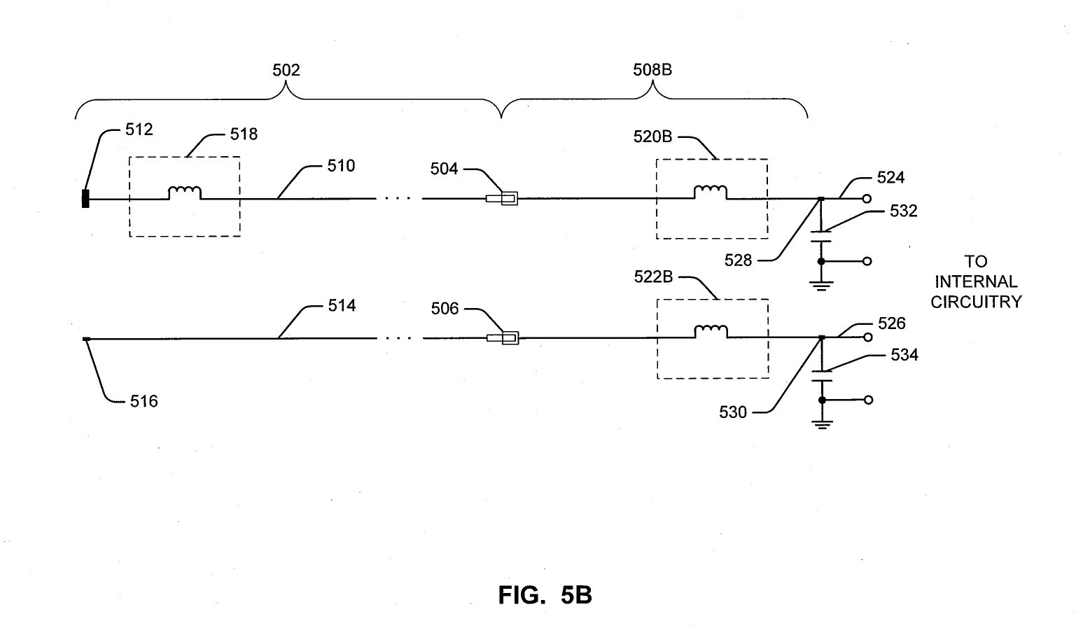

As mentioned above, a filter circuit as taught herein may be implemented in various ways and on one or more conductors of one or more leads. FIG. 5A is a simplified schematic diagram that illustrates that the filter circuits employed in a given case may comprise a combination of band stop filters and low pass filters, and that a different number of filter circuits may be employed in different implementations. Here, an implantable lead (corresponding to section 502) is coupled by connectors 504 and 506 to a device header (corresponding to section 508A).

The implantable lead includes a ring conductor 510 coupled to a ring electrode 512 and a tip conductor 514 coupled to a tip electrode 516. A low pass filter circuit 518 is deployed at a distal portion of the ring conductor 510 near the ring electrode 512. A filter circuit is not provided on the tip conductor 514 in this example.

The device header includes an L-C tank circuit 520A for the ring conductor 510 and an L-C tank circuit 522A for the tip conductor 514. The L-C tank circuits 520A and 522A are coupled to internal circuitry of an implantable medical device via conductors 524 and 526 that pass through respective feedthroughs 528 and 530. Here, the feedthroughs 528 and 530 have associated feedthrough capacitors 532 and 534.

FIG. 5A also illustrates an example where the filter circuits in the header are electrically isolated from one another. In this way, the dissipation of any MRI-induced heating may be further distributed throughout the header.

FIG. 5B is a simplified schematic diagram of an embodiment that employs broad-band filters in the device header. An implantable lead (corresponding to section 502) is coupled by connectors 504 and 506 to a device header (corresponding to section 508B). The device header includes a broad-band filter circuit 520B (e.g., an inductor) for the ring conductor 510 and a broad-band filter circuit 522B (e.g., an inductor) for the tip conductor 514. The broad-band filter circuits 520B and 522B are coupled to internal circuitry of an implantable medical device via conductors 524 and 526 that pass through respective feedthroughs 528 and 530. It should be appreciated based on the teachings herein that different combination of the filter components of FIGS. 5A and 5B (and/or any other filter components described herein) may be employed in different implementations. For example, a device header may employ a band stop filter for one conductor and a broad-band filter for another conductor in some implementations.

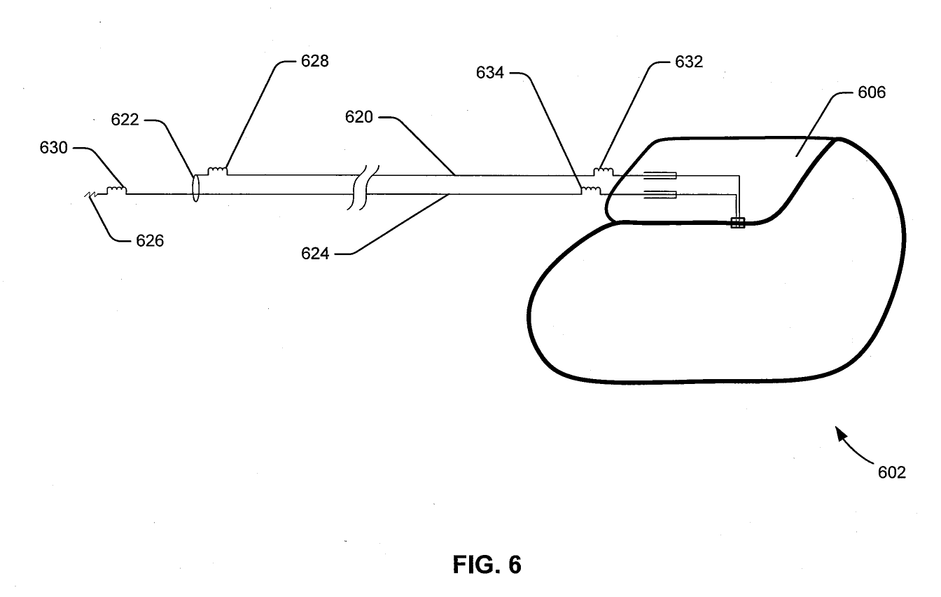

In some implementations, filter circuits are employed at the distal and proximal ends of an implantable lead. FIG. 6 illustrates a simplified schematic diagram where an implantable medical device 602 is connected to a lead that employs filter circuits on each end of two lead conductors. In this example, a filter circuit 628 is provided on a distal end of the lead near the ring electrode 622 for the ring conductor 620, and a filter circuit 630 is provided on a distal end of the lead near the tip electrode 626 for the tip conductor 624. In addition, a filter circuit 632 is provided on the ring conductor 620 at a proximal end of the lead (i.e., near the device 602) and a filter circuit 634 is provided on the tip conductor 624 at a proximal end of the lead.

In the implementation of FIG. 6, MRI-induced energy carried by the ring conductor 620 is attenuated by the filter circuit 628 prior to entering the ring electrode 622. Similarly, MRI-induced energy carried by the tip conductor 624 is attenuated by the filter circuit 630 prior to entering the tip electrode 626. Thus, heating of tissue at the electrode implant locations will be significantly reduced or entirely eliminated. Moreover, the MRI-induced energy will not cause significant heating or rectification at the device 602 due to the operation of the filter circuit 632 and the filter circuit 634 which will significantly attenuate this energy (e.g., as discussed above) prior to this energy entering the header 606 of the device 602.

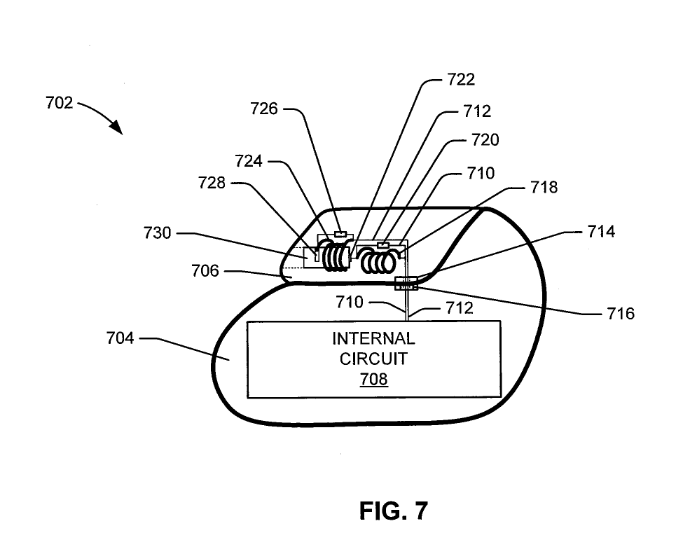

FIG. 7 illustrates another example of how filter circuits may be deployed within a header between a feedthrough and a lead connector. In particular, FIG. 7 illustrates an implementation where an inductor is wrapped around another component within the header. In this way, a sufficiently large inductance may be provided while keeping the size of the header as small as possible.

In this example, an implantable medical device 702 comprises a main body 704 and a header 706. A hermetically sealed feedthrough 708 is provided in the housing of the main body 704. An internal circuit 708 within the main body 704 is coupled to conductors 710 and 712 that pass through a feedthrough 714 into an interior space of the header 706. Here, a feedthrough capacitor 716 is implemented within the feedthrough 714 (e.g., around the conductors 710 and 712).

The conductor 710 is coupled to terminals of an inductor 718 and a capacitor 720 that form a first filter circuit. The other terminals of the inductor 718 and the capacitor 720 are coupled to a first terminal 722 of a connector 730 in the header 706.

The conductor 712 is coupled to terminals of an inductor 724 and a capacitor 726 that form a second filter circuit. The other terminals of the inductor 724 and the capacitor 726 are coupled to a second terminal 728 of the connector 730. Here, it may be seen that the inductor 724 is wound around a section of the connector 730.

The filter circuits described herein may be constructed in a variety of ways depending on the requirements of a given implementation. For example, it may be anticipated that a patient with an implantable device may be subjected to external rescue shocks. Thus, filters employing coil inductors may be designed to withstand high currents that may occur during such shocks. For example, in some implementations, a coil inductor comprises 3 mil diameter Drawn Filled Tube (DFT) wire (e.g., 41% Ag to 75% Ag) to withstand current on the order of 8 amps for a 2 millisecond pulse duration during an external defibrillation rescue shock.

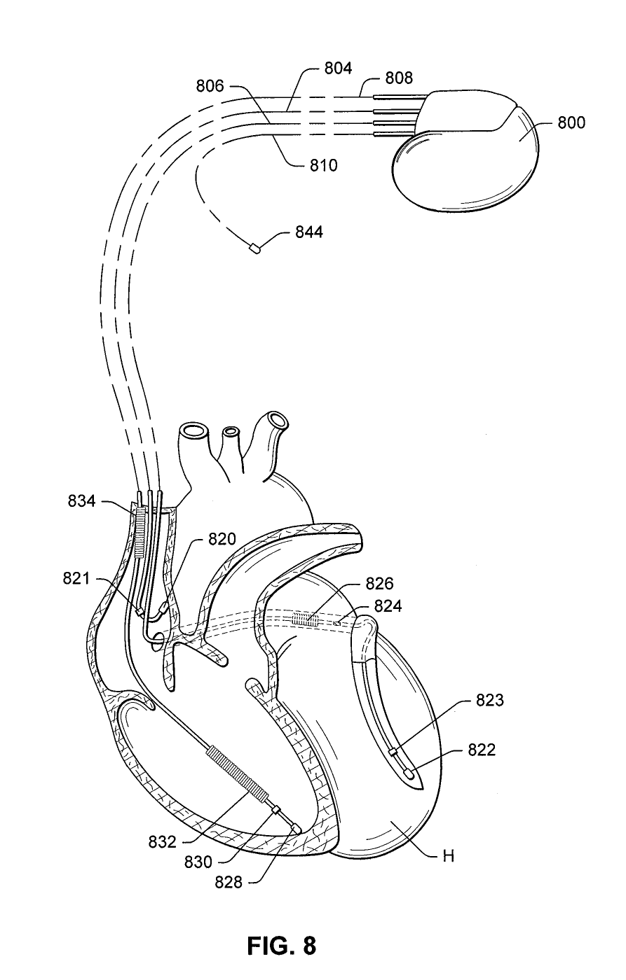

FIGS. 8 and 9 describe an exemplary implantable medical device (e.g., a stimulation device such as a pacemaker, an implantable cardioverter defibrillator, etc.) that is capable of being used in connection with the various embodiments that are described herein. It is to be appreciated and understood that other devices, including those that are not necessarily implantable, can be used and that the description below is given, in its specific context, to assist the reader in understanding, with more clarity, the embodiments described herein.

FIG. 8 shows an exemplary implantable cardiac device 800 in electrical communication with a patient's heart H by way of three leads 804, 806, and 808, suitable for delivering multi-chamber stimulation and shock therapy. Bodies of the leads 804, 806, and 808 may be formed of silicone, polyurethane, plastic, or similar biocompatible materials to facilitate implant within a patient. Each lead includes one or more conductors, each of which may couple one or more electrodes incorporated into the lead to a connector on the proximal end of the lead. Each connector, in turn, is configured to couple with a complimentary connector (e.g., implemented within a header) of the device 800.

To sense atrial cardiac signals and to provide right atrial chamber stimulation therapy, the device 800 is coupled to an implantable right atrial lead 804 having, for example, an atrial tip electrode 820, which typically is implanted in the patient's right atrial appendage or septum. FIG. 8 also shows the right atrial lead 804 as having an optional atrial ring electrode 821.

To sense left atrial and ventricular cardiac signals and to provide left chamber pacing therapy, the device 800 is coupled to a coronary sinus lead 806 designed for placement in the coronary sinus region via the coronary sinus for positioning one or more electrodes adjacent to the left ventricle, one or more electrodes adjacent to the left atrium, or both. As used herein, the phrase “coronary sinus region” refers to the vasculature of the left ventricle, including any portion of the coronary sinus, the great cardiac vein, the left marginal vein, the left posterior ventricular vein, the middle cardiac vein, the small cardiac vein or any other cardiac vein accessible by the coronary sinus.

Accordingly, an exemplary coronary sinus lead 806 is designed to receive atrial and ventricular cardiac signals and to deliver left ventricular pacing therapy using, for example, a left ventricular tip electrode 822 and, optionally, a left ventricular ring electrode 823; provide left atrial pacing therapy using, for example, a left atrial ring electrode 824; and provide shocking therapy using, for example, a left atrial coil electrode 826 (or other electrode capable of delivering a shock). For a more detailed description of a coronary sinus lead, the reader is directed to U.S. Pat. No. 5,466,254, “Coronary Sinus Lead with Atrial Sensing Capability” (Helland), which is incorporated herein by reference.

The device 800 is also shown in electrical communication with the patient's heart H by way of an implantable right ventricular lead 808 having, in this implementation, a right ventricular tip electrode 828, a right ventricular ring electrode 830, a right ventricular (RV) coil electrode 832 (or other electrode capable of delivering a shock), and a superior vena cava (SVC) coil electrode 834 (or other electrode capable of delivering a shock). Typically, the right ventricular lead 808 is transvenously inserted into the heart H to place the right ventricular tip electrode 828 in the right ventricular apex so that the RV coil electrode 832 will be positioned in the right ventricle and the SVC coil electrode 834 will be positioned in the superior vena cava. Accordingly, the right ventricular lead 808 is capable of sensing or receiving cardiac signals, and delivering stimulation in the form of pacing and shock therapy to the right ventricle.

The device 800 is also shown in electrical communication with a lead 810 including one or more components 844 such as a physiologic sensor. The component 844 may be positioned in, near or remote from the heart.

It should be appreciated that the device 800 may connect to leads other than those specifically shown. In addition, the leads connected to the device 800 may include components other than those specifically shown. For example, a lead may include other types of electrodes, sensors or devices that serve to otherwise interact with a patient or the surroundings.

FIG. 9 depicts an exemplary, simplified block diagram illustrating sample components of the device 800. The device 800 may be adapted to treat both fast and slow arrhythmias with stimulation therapy, including cardioversion, defibrillation, and pacing stimulation. While a particular multi-chamber device is shown, it is to be appreciated and understood that this is done for illustration purposes. Thus, the techniques and methods described below can be implemented in connection with any suitably configured or configurable device. Accordingly, one of skill in the art could readily duplicate, eliminate, or disable the appropriate circuitry in any desired combination to provide a device capable of treating the appropriate chamber(s) with, for example, cardioversion, defibrillation, and pacing stimulation.

A housing 900 for the device 800 is often referred to as the “can”, “case” or “case electrode”, and may be programmably selected to act as the return electrode for all “unipolar” modes. The housing 900 may further be used as a return electrode alone or in combination with one or more of the coil electrodes 826, 832 and 834 for shocking purposes. The housing 900 may be Constructed of a biocompatible material (e.g., titanium) to facilitate implant within a patient.

The housing 900 further includes a connector (not shown) having a plurality of terminals 901, 902, 904, 905, 906, 908, 912, 914, 916 and 918 (shown schematically and, for convenience, the names of the electrodes to which they are connected are shown next to the terminals). The connector may be configured to include various other terminals (e.g., terminal 921 coupled to a sensor or some other component) depending on the requirements of a given application.

To achieve right atrial sensing and pacing, the connector includes, for example, a right atrial tip terminal (AR TIP) 902 adapted for connection to the right atrial tip electrode 820. A right atrial ring terminal (AR RING) 901 may also be included and adapted for connection to the right atrial ring electrode 821. To achieve left chamber sensing, pacing, and shocking, the connector includes, for example, a left ventricular tip terminal (VL TIP) 904, a left ventricular ring terminal (VL RING) 905, a left atrial ring terminal (AL RING) 906, and a left atrial shocking terminal (AL COIL) 908, which are adapted for connection to the left ventricular tip electrode 822, the left ventricular ring electrode 823, the left atrial ring electrode 824, and the left atrial coil electrode 826, respectively.

To support right chamber sensing, pacing, and shocking, the connector further includes a right ventricular tip terminal (VR TIP) 912, a right ventricular ring terminal (VR RING) 914, a right ventricular shocking terminal (RV COIL) 916, and a superior vena cava shocking terminal (SVC COIL) 918, which are adapted for connection to the right ventricular tip electrode 828, the right ventricular ring electrode 830, the RV coil electrode 832, and the SVC coil electrode 834, respectively.

At the core of the device 800 is a programmable microcontroller 920 that controls the various modes of stimulation therapy. As is well known in the art, microcontroller 920 typically includes a microprocessor, or equivalent control circuitry, designed specifically for controlling the delivery of stimulation therapy, and may further include memory such as RAM, ROM and flash memory, logic and timing circuitry, state machine circuitry, and I/O circuitry. Typically, microcontroller 920 includes the ability to process or monitor input signals (data or information) as controlled by a program code stored in a designated block of memory. The type of microcontroller is not critical to the described implementations. Rather, any suitable microcontroller 920 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. No. 4,712,555 (Thornander et al.) and U.S. Pat. No. 4,944,298 (Sholder), all of which are incorporated by reference herein. For a more detailed description of the various timing intervals that may be used within the device and their inter-relationship, see U.S. Pat. No. 4,788,980 (Mann et al.), also incorporated herein by reference.

FIG. 9 also shows an atrial pulse generator 922 and a ventricular pulse generator 924 that generate pacing stimulation pulses for delivery by the right atrial lead 804, the coronary sinus lead 806, the right ventricular lead 808, or some combination of these leads via an electrode configuration switch 926. It is understood that in order to provide stimulation therapy in each of the four chambers of the heart, the atrial and ventricular pulse generators 922 and 924 may include dedicated, independent pulse generators, multiplexed pulse generators, or shared pulse generators. The pulse generators 922 and 924 are controlled by the microcontroller 920 via appropriate control signals 928 and 930, respectively, to trigger or inhibit the stimulation pulses.

Microcontroller 920 further includes timing control circuitry 932 to control the timing of the stimulation pulses (e.g., pacing rate, atrio-ventricular (A-V) delay, atrial interconduction (A-A) delay, or ventricular interconduction (V-V) delay, etc.) or other operations, as well as to keep track of the timing of refractory periods, blanking intervals, noise detection windows, evoked response windows, alert intervals, marker channel timing, etc., as known in the art.

Microcontroller 920 further includes an arrhythmia detector 934. The arrhythmia detector 934 may be utilized by the device 800 for determining desirable times to administer various therapies. The arrhythmia detector 934 may be implemented, for example, in hardware as part of the microcontroller 920, or as software/firmware instructions programmed into the device 800 and executed on the microcontroller 920 during certain modes of operation.

Microcontroller 920 may include a morphology discrimination module 936, a capture detection module 937 and an auto sensing module 938. These modules are optionally used to implement various exemplary recognition algorithms or methods. The aforementioned components may be implemented, for example, in hardware as part of the microcontroller 920, or as software/firmware instructions programmed into the device 800 and executed on the microcontroller 920 during certain modes of operation.

The electrode configuration switch 926 includes a plurality of switches for connecting the desired terminals (e.g., that are connected to electrodes, coils, sensors, etc.) to the appropriate I/O circuits, thereby providing complete terminal and, hence, electrode programmability. Accordingly, switch 926, in response to a control signal 942 from the microcontroller 920, may be used to determine the polarity of the stimulation pulses (e.g., unipolar, bipolar, combipolar, etc.) by selectively closing the appropriate combination of switches (not shown) as is known in the art.

Atrial sensing circuits (ATR. SENSE) 944 and ventricular sensing circuits (VTR. SENSE) 946 may also be selectively coupled to the right atrial lead 804, coronary sinus lead 806, and the right ventricular lead 808, through the switch 926 for detecting the presence of cardiac activity in each of the four chambers of the heart. Accordingly, the atrial and ventricular sensing circuits 944 and 946 may include dedicated sense amplifiers, multiplexed amplifiers, or shared amplifiers. Switch 926 determines the “sensing polarity” of the cardiac signal by selectively closing the appropriate switches, as is also known in the art. In this way, the clinician may program the sensing polarity independent of the stimulation polarity. The sensing circuits (e.g., circuits 944 and 946) are optionally capable of obtaining information indicative of tissue capture.

Each sensing circuit 944 and 946 preferably employs one or more low power, precision amplifiers with programmable gain, automatic gain control, bandpass filtering, a threshold detection circuit, or some combination of these components, to selectively sense the cardiac signal of interest. The automatic gain control enables the device 800 to deal effectively with the difficult problem of sensing the low amplitude signal characteristics of atrial or ventricular fibrillation.

The outputs of the atrial and ventricular sensing circuits 944 and 946 are connected to the microcontroller 920, which, in turn, is able to trigger or inhibit the atrial and ventricular pulse generators 922 and 924, respectively, in a demand fashion in response to the absence or presence of cardiac activity in the appropriate chambers of the heart. Furthermore, as described herein, the microcontroller 920 is also capable of analyzing information output from the sensing circuits 944 and 946, a data acquisition system 952, or both. This information may be used to determine or detect whether and to what degree tissue capture has occurred and to program a pulse, or pulses, in response to such determinations. The sensing circuits 944 and 946, in turn, receive control signals over signal lines 948 and 950, respectively, from the microcontroller 920 for purposes of controlling the gain, threshold, polarization charge removal circuitry (not shown), and the timing of any blocking circuitry (not shown) coupled to the inputs of the sensing circuits 944 and 946 as is known in the art.

For arrhythmia detection, the device 800 utilizes the atrial and ventricular sensing circuits 944 and 946 to sense cardiac signals to determine whether a rhythm is physiologic or pathologic. It should be appreciated that other components may be used to detect arrhythmia depending on the system objectives. In reference to arrhythmias, as used herein, “sensing” is reserved for the noting of an electrical signal or obtaining data (information), and “detection” is the processing (analysis) of these sensed signals and noting the presence of an arrhythmia.

Timing intervals between sensed events (e.g., P-waves, R-waves, and depolarization signals associated with fibrillation) may be classified by the arrhythmia detector 934 of the microcontroller 920 by comparing them to a predefined rate zone limit (e.g., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”). Similar rules may be applied to the atrial channel to determine if there is an atrial tachyarrhythmia or atrial fibrillation with appropriate classification and intervention.

Cardiac signals or other signals may be applied to inputs of an analog-to-digital (ND) data acquisition system 952. The data acquisition system 952 is configured (e.g., via signal line 956) to acquire intracardiac electrogram (“IEGM”) signals or other signals, convert the raw analog data into a digital signal, and store the digital signals for later processing, for telemetric transmission to an external device 954, or both. For example, the data acquisition system 952 may be coupled to the right atrial lead 804, the coronary sinus lead 806, the right ventricular lead 808 and other leads through the switch 926 to sample cardiac signals across any pair of desired electrodes.

The data acquisition system 952 also may be coupled to receive signals from other input devices. For example, the data acquisition system 952 may sample signals from a physiologic sensor 970 or other components shown in FIG. 9 (connections not shown).

The microcontroller 920 is further coupled to a memory 960 by a suitable data/address bus 962, wherein the programmable operating parameters used by the microcontroller 920 are stored and modified, as required, in order to customize the operation of the device 800 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart H within each respective tier of therapy. One feature of the described embodiments is the ability to sense and store a relatively large amount of data (e.g., from the data acquisition system 952), which data may then be used for subsequent analysis to guide the programming of the device 800.

Advantageously, the operating parameters of the implantable device 800 may be non-invasively programmed into the memory 960 through a telemetry circuit 964 in telemetric communication via communication link 966 with the external device 954, such as a programmer, transtelephonic transceiver, a diagnostic system analyzer or some other device. The microcontroller 920 activates the telemetry circuit 964 with a control signal (e.g., via bus 968). The telemetry circuit 964 advantageously allows intracardiac electrograms and status information relating to the operation of the device 800 (as contained in the microcontroller 920 or memory 960) to be sent to the external device 954 through an established communication link 966.

The device 800 can further include one or more physiologic sensors 970. In some embodiments the device 800 may include a “rate-responsive” sensor that may provide, for example, information to aid in adjustment of pacing stimulation rate according to the exercise state of the patient. One or more physiologic sensors 970 (e.g., a pressure sensor) may further be used to detect changes in cardiac output, changes in the physiological condition of the heart, or diurnal changes in activity (e.g., detecting sleep and wake states). Accordingly, the microcontroller 920 responds by adjusting the various pacing parameters (such as rate, A-V Delay, V-V Delay, etc.) at which the atrial and ventricular pulse generators 922 and 924 generate stimulation pulses.

While shown as being included within the device 800, it is to be understood that a physiologic sensor 970 may also be external to the device 800, yet still be implanted within or carried by the patient. Examples of physiologic sensors that may be implemented in conjunction with the device 800 include sensors that sense respiration rate, pH of blood, ventricular gradient, oxygen saturation, blood pressure and so forth. Another sensor that may be used is one that detects activity variance, wherein an activity sensor is monitored diurnally to detect the low variance in the measurement corresponding to the sleep state. For a more detailed description of an activity variance sensor, the reader is directed to U.S. Pat. No. 5,476,483 (Bornzin et al.), which patent is hereby incorporated by reference.

The one or more physiologic sensors 970 may optionally include one or more of components to help detect movement (via, e.g., a position sensor or an accelerometer) and minute ventilation (via an MV sensor) in the patient. Signals generated by the position sensor and MV sensor may be passed to the microcontroller 920 for analysis in determining whether to adjust the pacing rate, etc. The microcontroller 920 may thus monitor the signals for indications of the patient's position and activity status, such as whether the patient is climbing up stairs or descending down stairs or whether the patient is sitting up after lying down.

The device 800 additionally includes a battery 976 that provides operating power to all of the circuits shown in FIG. 9. For a device 800 which employs shocking therapy, the battery 976 is capable of operating at low current drains (e.g., preferably less than 10 μA) for long periods of time, and is capable of providing high-current pulses (for capacitor charging) when the patient requires a shock pulse (e.g., preferably, in excess of 2 A, at voltages above 200 V, for periods of 10 seconds or more). The battery 976 also desirably has a predictable discharge characteristic so that elective replacement time can be detected. Accordingly, the device 800 preferably employs lithium or other suitable battery technology.

The device 800 can further include magnet detection circuitry (not shown), coupled to the microcontroller 920, to detect when a magnet is placed over the device 800. A magnet may be used by a clinician to perform various test functions of the device 800 and to signal the microcontroller 920 that the external device 954 is in place to receive data from or transmit data to the microcontroller 920 through the telemetry circuit 964.

The device 800 further includes an impedance measuring circuit 978 that is enabled by the microcontroller 920 via a control signal 980. The known uses for an impedance measuring circuit 978 include, but are not limited to, lead impedance surveillance during the acute and chronic phases for proper performance, lead positioning or dislodgement; detecting operable electrodes and automatically switching to an operable pair if dislodgement occurs; measuring respiration or minute ventilation; measuring thoracic impedance for determining shock thresholds; detecting when the device 800 has been implanted; measuring stroke volume; and detecting the opening of heart valves, etc. The impedance measuring circuit 978 is advantageously coupled to the switch 926 so that any desired electrode may be used.

In the case where the device 800 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 920 further controls a shocking circuit 982 by way of a control signal 984. The shocking circuit 982 generates shocking pulses of low (e.g., up to 0.5 J), moderate (e.g., 0.5 J to 10 J), or high energy (e.g., 11 J to 40 J), as controlled by the microcontroller 920. Such shocking pulses are applied to the patient's heart H through, for example, two shocking electrodes and as shown in this embodiment, selected from the left atrial coil electrode 826, the RV coil electrode 832 and the SVC coil electrode 834. As noted above, the housing 900 may act as an active electrode in combination with the RV coil electrode 832, as part of a split electrical vector using the SVC coil electrode 834 or the left atrial coil electrode 826 (i.e., using the RV electrode as a common electrode), or in some other arrangement.

Cardioversion level shocks are generally considered to be of low to moderate energy level (so as to minimize pain felt by the patient), be synchronized with an R-wave, pertain to the treatment of tachycardia, or some combination of the above. Defibrillation shocks are generally of moderate to high energy level (i.e., corresponding to thresholds in the range of 5 J to 40 J), delivered asynchronously (since R-waves may be too disorganized), and pertaining to the treatment of fibrillation. Accordingly, the microcontroller 920 is capable of controlling the synchronous or asynchronous delivery of the shocking pulses.

The filter circuits described herein may be implemented at or near one or more of the components of FIG. 9. For example, a filter circuit may be implanted at or near the connector or the switch 926.

Various modifications may be incorporated into the disclosed embodiments based on the teachings herein. For example, the structure and functionality taught herein may be incorporated into types of devices other than the specific types of devices described above. In addition, different filtering components and filtering schemes may be employed consistent with the teachings herein.

The various structures and functions described herein may be incorporated into a variety of apparatuses (e.g., a stimulation device, a lead, a monitoring device, etc.) and implemented in a variety of ways. Different embodiments of such an apparatus may include a variety of hardware and software processing components. In some embodiments, hardware components such as processors, controllers, state machines, logic, or some combination of these components, may be used to implement the described components or circuits.

In some embodiments, code including instructions (e.g., software, firmware, middleware, etc.) may be executed on one or more processing devices to implement one or more of the described functions or components. The code and associated components (e.g., data structures and other components used by the code or used to execute the code) may be stored in an appropriate data memory that is readable by a processing device (e.g., commonly referred to as a computer-readable medium).

Moreover, some of the operations described herein may be performed by a device that is located externally with respect to the body of the patient. For example, an implanted device may send raw data or processed data to an external device that then performs the necessary processing.

The components and functions described herein may be connected or coupled in many different ways. The manner in which this is done may depend, in part, on whether and how the components are separated from the other components. In some embodiments some of the connections or couplings represented by the lead lines in the drawings may be in an integrated circuit, on a circuit board or implemented as discrete wires or in other ways.

As used herein, terminology describing the coupling of components refers to any mechanism that allows signals to travel from one component to another. Thus, coupling may be accomplished through use of an electrical conductor and/or an electrical component (e.g., an active or passive electrical circuit). In some cases two or more components may be “directly coupled.” That is, the components may be coupled via a conductor without any intervening components (e.g., an active or passive electrical circuit) between the components. Also, the term circuit is used herein in a broad sense of a component or components through which current may flow, and is not limited to the narrower definition of a structure that forms a loop. For example, a circuit may comprise one component (e.g., a conductor, an electronic component, etc.) or more than one component (e.g., several electronic components connected by one or more conductors). As a specific example, a ground circuit may take the form of a single conductor, a ground plane, multiple conductors, multiple ground planes, or some other form. As another specific example, an electrode circuit may take the form of a single conductor, a conductor and a connector, multiple conductors, or some other form.

The signals discussed herein may take various forms. For example, in some embodiments a signal may comprise electrical signals transmitted over a wire, light pulses transmitted through an optical medium such as an optical fiber or air, or RF waves transmitted through a medium such as air, and so on. In addition, a plurality of signals may be collectively referred to as a signal herein. The signals discussed above also may take the form of data. For example, in some embodiments an application program may send a signal to another application program. Such a signal may be stored in a data memory.

Moreover, the recited order of the blocks in any processes disclosed herein is simply an example of a suitable approach. Thus, operations associated with such blocks may be rearranged while remaining within the scope of the present disclosure. Similarly, any accompanying method claims present operations in a sample order, and are not necessarily limited to the specific order presented.

Also, it should be understood that any reference to elements herein using a designation such as “first,” “second,” and so forth does not generally limit the quantity or order of those elements. Rather, these designations may be used herein as a convenient method of distinguishing between two or more different elements or instances of an element. Thus, a reference to first and second elements does not mean that only two elements may be employed there or that the first element must precede the second element in some manner. Also, unless stated otherwise a set of elements may comprise one or more elements. In addition, terminology of the form “at least one of A, B, or C” or “one or more of A, B, or C” or “at least one of the group consisting of A, B, and C” used in the description or the claims means “A or B or C or any combination of these elements.”

While certain embodiments have been described above in detail and shown in the accompanying drawings, it is to be understood that such embodiments are merely illustrative of and not restrictive of the teachings herein. In particular, it should be recognized that the teachings herein apply to a wide variety of apparatuses and methods. It will thus be recognized that various modifications may be made to the illustrated embodiments or other embodiments, without departing from the broad scope thereof. In view of the above it will be understood that the teachings herein are intended to cover any changes, adaptations or modifications which are within the scope of the disclosure.

What is claimed is:

1. An implantable medical device, comprising:
a hermetically sealed housing for electronic circuitry, wherein the hermetically sealed housing comprises a hermetically sealed feedthrough;
a header housing mounted to the hermetically sealed housing over the feedthrough;
a first conductor coupled to the electronic circuitry and routed through the feedthrough such that the first conductor passes into an interior space defined by the header housing;
a feedthrough capacitor coupled to the first conductor in proximity to the feedthrough;
at least one connector mounted within the header housing; and
a first filter circuit mounted within the header housing and coupled between a first terminal of the at least one connector and the first conductor, wherein:
the first filter circuit is electrically isolated from the header housing; and
an impedance of the first filter circuit at an MRI scanning frequency is at least an order of magnitude greater than an impedance of the feedthrough capacitor at the MRI scanning frequency.

2. The device of claim 1, wherein the impedance of the first filter circuit is at least 200 ohms at 64 MHz and at 128 MHz.

3. The device of claim 1, further comprising:
a second conductor coupled to the electronic circuitry and routed through the feedthrough such that the second conductor passes into the interior space defined by the header housing; and
a second filter circuit mounted within the header housing and coupled between a second terminal of the at least one connector and the second conductor, wherein:
the second filter circuit is electrically isolated from the header housing and the first filter circuit; and
an impedance of the second filter circuit at the MRI scanning frequency is at least an order of magnitude greater than the impedance of the feedthrough capacitor at the MRI scanning frequency.

4. The device of claim 3, wherein:
the impedance of the first filter circuit is at least 200 ohms at 64 MHz and at 128 MHz; and
the impedance of the second filter circuit is at least 200 ohms at 64 MHz and at 128 MHz.

5. The device of claim 3, wherein:
the first filter circuit is a band stop filter; and
the second filter circuit is a band stop filter.

6. The device of claim 5, wherein each band stop filter has a resonant frequency that corresponds to an MRI scanning frequency.

7. The device of claim 6, wherein each resonant frequency is approximately 64 MHz or approximately 128 MHz.

8. The device of claim 5, wherein each band stop filter comprises an L-C tank circuit.

9. The device of claim 3, wherein:
the first filter circuit is a broad-band filter; and
the second filter circuit is a broad-band filter.

10. The device of claim 3, wherein:
the first filter circuit is a low pass filter; and
the second filter circuit is a low pass filter.

11. The device of claim 3, wherein:
the first filter circuit is an inductor; and
the second filter circuit is an inductor.

12. The device of claim 11, wherein at least one of the first inductor and the second inductor is wound around a section of the at least one connector.

13. An implantable medical system, comprising:
an implantable lead comprising:
a first conductor;
a first electrode coupled to the first conductor; and
a first filter circuit proximate to the first electrode and coupled in series with the first conductor; and
an implantable medical device, comprising:
a hermetically sealed housing for electronic circuitry, wherein the hermetically sealed housing comprises a hermetically sealed feedthrough;
a header housing mounted to the hermetically sealed housing over the feedthrough;
a second conductor coupled to the electronic circuitry and routed through the feedthrough such that the second conductor passes into an interior space defined by the header housing; and
a feedthrough capacitor coupled to the second conductor in proximity to the feedthrough;
a connector mounted within the header housing and comprising a first terminal configured to contact the first conductor; and
a second filter circuit mounted within the header housing and coupled between the first terminal of the connector and the second conductor, wherein:
the second filter circuit is electrically isolated from the header housing; and
an impedance of the second filter circuit at an MRI scanning frequency is at least an order of magnitude greater than an impedance of the feedthrough capacitor at the MRI scanning frequency.

14. The system of claim 13, wherein the first filter circuit is a band stop filter that has a resonant frequency that corresponds to an MRI scanning frequency.

15. The system of claim 14, wherein the resonant frequency is approximately 64 MHz or approximately 128 MHz.

16. The system of claim 13, wherein the second filter circuit is a band stop filter that has a resonant frequency that corresponds to the MRI scanning frequency.

17. The system of claim 13, wherein the second filter circuit is a low pass filter.

18. The system of claim 13, wherein the second filter circuit is an inductor.

19. The system of claim 18, wherein the inductor is wound around a section of the at least one connector.

20. The system of claim 13, wherein:
the implantable lead further comprises a third conductor;
the connector further comprises a second terminal configured to contact the third conductor;
the implantable medical device further comprises:
a fourth conductor coupled to the electronic circuitry and routed through the feedthrough such that the fourth conductor passes into the interior space defined by the header housing; and
a third filter circuit mounted within the header housing and coupled between the second terminal of the connector and the fourth conductor;
the third filter circuit is electrically isolated from the header housing and the second filter circuit; and
an impedance of the third filter circuit at the MRI scanning frequency is at least an order of magnitude greater than the impedance of the feedthrough capacitor at the MRI scanning frequency.
